(19) United States
(12) Patent Application Publication
(10) Pub. No.: US 2018/0297858 A1
(43) Pub. Date: Oct. 18, 2018

(54) METHODS AND COMPOSITIONS RELATED TO MAGNETO-ELASTO-ELECTROPORATION (MEEP)

(71) Applicants: Soutik BETAL, San Antonio, TX (US); Binita SHRESTHA, San Antonio, TX (US); Moumita DUTTA, San Antonio, TX (US); Kelly NASH, San Antonio, TX (US); Liang TANG, San Antonio, TX (US); Amar S BHALLA, San Antonio, TX (US); Ruyan GUO, San Antonio, TX (US)

(72) Inventors: Soutik BETAL, San Antonio, TX (US); Binita SHRESTHA, San Antonio, TX (US); Moumita DUTTA, San Antonio, TX (US); Kelly NASH, San Antonio, TX (US); Liang TANG, San Antonio, TX (US); Amar S BHALLA, San Antonio, TX (US); Ruyan GUO, San Antonio, TX (US)

(73) Assignee: THE BOARD OF REGENTS OF THE UNIVERSITY OF TEXAS SYSTEM, Austin, TX (US)

(21) Appl. No.: 15/768,141

(22) PCT Filed: Oct. 14, 2016
(86) PCT No.: PCT/US2016/057037

§ 371 Date: Apr. 13, 2018

Related U.S. Application Data
(60) Provisional application No. 62/241,786, filed on Oct. 15, 2015.

(51) Int. Cl. C01G 51/00; H01F 1/00; A61K 9/00; A61K 9/51; H01F 1/34

(52) U.S. Cl. CPC C01G 51/006; H01F 1/0054; A61K 9/0009; A61K 9/5115; H01F 1/344; B82Y 5/00; C01P 2002/84; C01P 2002/85; C01P 2004/04; C01P 2004/64; C01P 2002/72

(57) ABSTRACT

Embodiments of the invention are directed to Magneto-Elasto-Electroporation (MEEP) effect by manipulating cell electroporation induced by core shell magnetoelectric nanoparticles (CSMEN).

### CROSS-REFERENCE TO RELATED APPLICATIONS

This application claim the benefit of priority of U.S. provisional patent application No. 62/241,786, filed Oct. 15, 2015, which is hereby incorporated by reference in its entirety.

### BACKGROUND OF THE INVENTION

### Field of the Invention

The present invention relates generally to the field of cell biology and molecular biology. More particularly, it concerns nanoparticles and method of delivering moieties across a cell membrane.

### Description of Related Art

Electroporation is a well-defined phenomenon that is experimentally validated and mathematically defined in the scientific literature. Electroporation is the physical phenomenon which results in the transient loss of semi permeability of the cell membrane when exposed to microsecond to nanoseconds electric pulse of sufficient intensity (Tsong, 1991). The membrane specific conductance for cell membrane is usually <10⁻³ S cm⁻¹ under normal physiological condition. An applied transmembrane electric field above the critical value that is normally 0.2-1 V, significantly increases the membrane specific conductance up to 1 S cm⁻¹ within microsecond resulting in the rearrangement of the lipid bi-layer. The electric dipoles of the lipid molecules reorient themselves in presence of electric field creating aqueous pores. Furthermore the finite permeability of the lipid bi-layer allows current to flow through the bi-layer resulting in a thermal phase transition of the lipid bi-layer. Both of these events give rise to conformational changes in the cell membrane thereby increasing membrane permeability to ions, molecules, and macromolecules (Chen, 2006). Donald et al reported formation of volcano shaped membrane openings of 20-120 nm diameters within 20 ms of applied electric field (Chang, 1990). This increase in cell permeability has been successfully employed for over a decade for DNA or gene transfection, protein insertion, cell fusion, enhanced uptake of metallic nanoparticle, or improved drug delivery (Chang, 1990). During the electroporation process nanopores open allowing sodium and/or potassium ions flow in or out of the cell until equilibrium between cell's internal and external potential is reached (Khaja Mohaideen and Joy, Journal of Magnetism and Magnetic Materials, 2014, 371:121-29; Sablik, 2002. 615:1613-20; du Tremolet de Lacheisserie, E., J. Magn. Magn. Mater, 1982, 25:251-70; Liang and Prorok, Appl. Phys. Lett, 2007, 90:221912; Landau, L. D. L., E.M. Theory of Elasticity. Pergamon Press: New York, N.Y., USA, 1986. 3rd ed.).

There remains a need for addition methods and techniques for delivery of various molecules to cell.

### SUMMARY OF THE INVENTION

Certain embodiments are directed to Magneto-elasto-electroporation (MEEP), which is a phenomenon where nanopores open in a cell membrane due to interaction with core shell magnetoelectric nanoparticles under the influence of ac magnetic field. Embodiments of the invention use a core-shell magnetoelectric nanoparticle (CSMEN) comprising a magnetostricitve core and a ferroelectric shell to achieve MEEP across cell membranes. The core of the CSMEN is encapsulated by piezoelectric shell. The encapsulated core is capable of producing a photoacoustic emission and/or a magnetoelastic emission under influence of alternating current (AC) magnetic field. The core of the CSMEN will experience strain in the form of expansion and contraction in presence of an AC magnetic field. The strain on the CSMEN core will generate a magnetoelastic wave that is absorbed by the shell as pressure wave. The absorbing of the pressure wave changes the surface potential due to the shell's piezoelectric property. The continuous change of surface potential of CSMENs under influence of AC magnetic field results in a transmembrane voltage change across a lipid membrane when CSMENs are positioned nanometers from lipid membrane. This transmembrane voltage result in opening of nanopores on cell membrane. The CSMENs will penetrate the lipid membrane through these electrically opened nanopores due to the magnetic moment of CSMENs towards magnets. In certain aspects the CSMENs can be exposed to an AC magntic field for a period of time sufficient for CSMENs to penetrate and pass through multiple lipid membranes, e.g., from one cell to another. In certain aspects the frequency and amplitude of the AC magnetic field can be optimized for various lipid membrane compositions, i.e., for different cell or tissue types.

In certain aspects the core comprises cobalt ferrite CoFe₂O₄. The core can be substituted with transition metal (M), e.g. Co₁₋ₓMₓFe₂O₄ where x<0.1 g/ml. The core can be used to form a biocompatible and non-cytotoxic (as tested with MTS assay) nanoparticle. In certain aspects the core is a single crystalline CoFe₂O₄ core.

The shell is a piezoelectric shell and can have a single crystalline or tetragonal perovskite structure. In certain aspects the shell is a BaTiO₃ shell. The BaTiO₃ can be substituted with strontium or magnesium, e.g. SrBaTiO₃, MgBaTiO₃. The shell forms a biocompatible and non-cytoxic nanoparticle as determined by MTS assay.

Certain embodiments of the invention are directed to method for achieving Magneto-elasto-electroporation (MEEP). In certain aspects the methods comprise (i) contacting a lipid membrane with CSMEN particles described herein; (ii) exposing the lipid membrane and CSMEN particles to an alternating current (AC) magnetic field across the lipid membrane. In certain aspects AC magnetic field has an intensity between 50 to 100 Oe and a frequency between 20 to 100 Hz. In certain aspects exposure to the AC magnetic field for at least, at most, or about 1, 10, 20, 30, 40, 50, or 60 second time intervals (including all values and ranges there between) for between 1, 10, 20, or 30 to 30, 40, 50, or 60 minutes (including all values and ranges there between). In certain aspects the MEEP can be performed in conjunction with imaging or locating the positions of the nanoparticles.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1F—Schematics of the MEEP mechanism. FIG. 1A, the area of cell membrane in nanometers having a pore presence probability is considered flat. Particles are positioned near the cell membrane. Uᵢₙₜ is showing the internal potential and Uₑₓₜ is the outside potential of the cell and particles present in external vicinity of the cell changes the Uₑₓₜ as AC magnetic field is turned on. On the other side of the cell is an electro magnet which attracts the CSMEN towards itself. FIG. 1B shows the CSMEN are nanometers from Cell membrane. FIG. 1C shows the strain in the core of nanoparticle due at a given magnetic field. FIG. 1D shows the particle with transparent shell and dense core with shell as invisible to show the opened nanopore. FIG. 1E Shows the particles just penetrating the membrane into the cell through electrically opened nanopore. FIG. 1F shows particle penetrates through the membrane into the cell towards magnets due to magnetic moment of the particles.

FIGS. 2A-2C—Transmission Electron Microscopy (TEM) images—Diffraction pattern and energy dispersive X-ray spectroscopy (EDS): FIG. 2A TEM image confirming Core-Shell structure confirmation. Diffraction Pattern illustrates the single crystalline nature of the BaTiO₃ shell. FIG. 2B: TEM image of cobalt ferrite nanoparticles and Diffraction Pattern illustrates the single crystalline nature of the core. FIG. 2C Energy Dispersive X-Ray analysis shows energy peaks of Barium, Titanium, Cobalt, Iron and Oxygen peaks.

FIGS. 3A-3E—Cobalt ferrite nanoparticles and CSMEN structural characteristics. FIG. 3A Atomic force Microscopy (AFM) image-Coated Nanoparticles, FIG. 3B Meta-ZetaSizer—curve of size analysis of cobalt ferrite nanoparticles. FIG. 3C Data of size measurement of BaTiO3 coated Cobalt Ferrite nanoparticles (CSMEN). FIG. 3D AFM scanning-shows the scanning of cobalt ferrite nanoparticles, data scale of the first image is (˜50 to 50 nm) and size in the 1^{st} image is about 50-55 nm whereas the second image of CSMEN, data scale (˜100 to 100 nm) shows the size of CSMEN as 75-79 nm. FIG. 3E AFM Scanning topography image shows the size as described in FIG. 3D.

FIGS. 4A-4D—Measurements confirming Magnetoelectric Emission from CSMEN, FIG. 4A Piezo Response Force Microscopy (PFM) measurements—The phase transition when biased voltage of +10V and −10V is applied whereas no phase change can be seen while applying 0V, FIG. 4B Hysteresis Curve—The Magnetometer results shows that cobalt ferrite nanoparticles have 51 emu/g magnetization whereas after coating with different amount of Barium titanate the magnetization decreases to 22 emu/g with 60% CFO-40% BT and 18.4 emu/g with 50% CFO-50% BT. FIG. 4C and FIG. 4D Opto-Acoustic Emission graph and data respectively—Photoacoustic emission peak intensity of cobalt ferrite nanoparticles decreases when AC Magnetic field is applied and further when CSMEN were analyzed the OA intensity peak further reduces.

FIGS. 5A-5E—Longitudinal Penetration Analysis. FIG. 5A The particles added on one end of the glass side in the well plate and magnetic field is applied from the other end. Since the HEP2 cells are in between particles and cells, the AC field will create the MEEP that causes the particle to penetrate through the electrically opened nanopores. FIG. 5B shows a schematic of the experiment. FIG. 5C Fluorescence microscope image in R mode to show the Cells cytoplasm stained with cell mask which is a Red Dye and; FIG. 5D shows Fluorescence microscope images in G mode where the nanoparticle loaded with FITC dye which stains green at the same spot where image was taken in R mode. FIG. 5E UV Vis spectrophotometer result of CSMEN at different compositions, silica coating on CSMEN and FITC loading on silica coated CSMEN is confirmed since FITC dye has a peak absorbance at 500-520 nm.

FIGS. 6A-6I—Fluorescence and Confocal Microscopy Images: FIG. 6A Fluorescence microscopy image of HEP2 cells with CSMEN exposed to DC magnetic field in R mode, FIG. 6B Refection of green fluorescence can be clearly seen on outside of Cell membrane in the Fluorescence microscopy image of HEP2 cells with CSMEN exposed to DC magnetic field in G mode FIG. 6C: Image 6(a), 6(b) merged together using ImageJ software. In the images it can be clearly seen that the particles are attached to the outside of cell membrane. FIG. 6D Fluorescence microscopy image of HEP2 cells with CSMEN exposed to AC magnetic field in R mode, FIG. 6E Particles penetrated into the HEP2 and scatters the green fluorescence inside the membrane and refection can be easily seen from the inside of the cell as seen in the Fluorescence microscopy image of HEP2 cells with CSMEN exposed to AC magnetic field in G mode, FIG. 6F: Image 6D and 6E merged together using ImageJ software. In the images it can be clearly seen that the CSMEN penetrates into the HEP2 Cells. FIG. 6G, FIG. 6H, and FIG. 6I Confirmation of CSMEN penetration into HEP2 using Confocal Microscopy done on the slides and particle penetration can be seen inside the HEP2 cell since optical slicing was done on the Confocal Microscopy images and slices in nanometer scale were cut from the cell.

FIGS. 7A-7B—Schematics and Data of the Transwell measurements. FIG. 7A Diagram of Transwell experiment, FIG. 7B Trans-well graph shows increased filtrate intensity over time in presence of AC Magnetic field whereas the fluorescence intensity of filtrate in case of DC field and control remains minimal.

FIG. 8—Cytotoxicity test Data: MTS assay was performed for different composition of CSMEN.

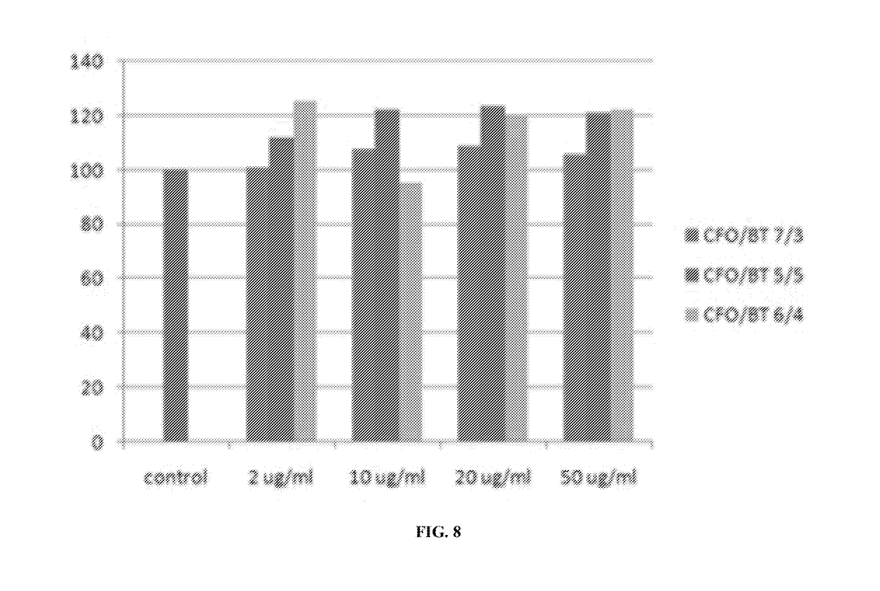

FIG. 9—Time Dependent Cytotoxicity test—In AC and DC field with 50<g/ml concentration.

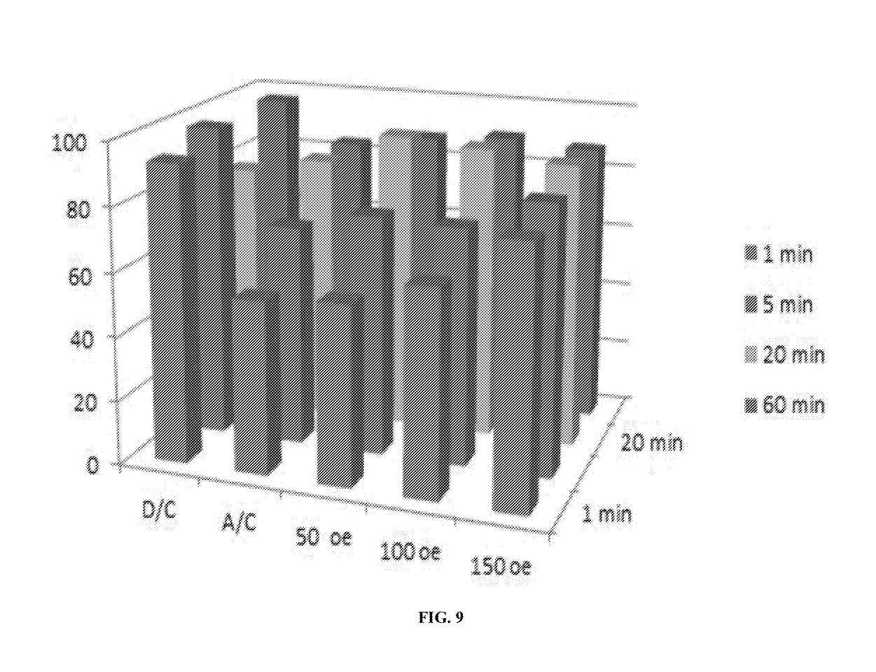

FIG. 10—Nanopore seen on the cell membrane of the fixed HEP2 Cell & Particle penetration—Bright Field Image.

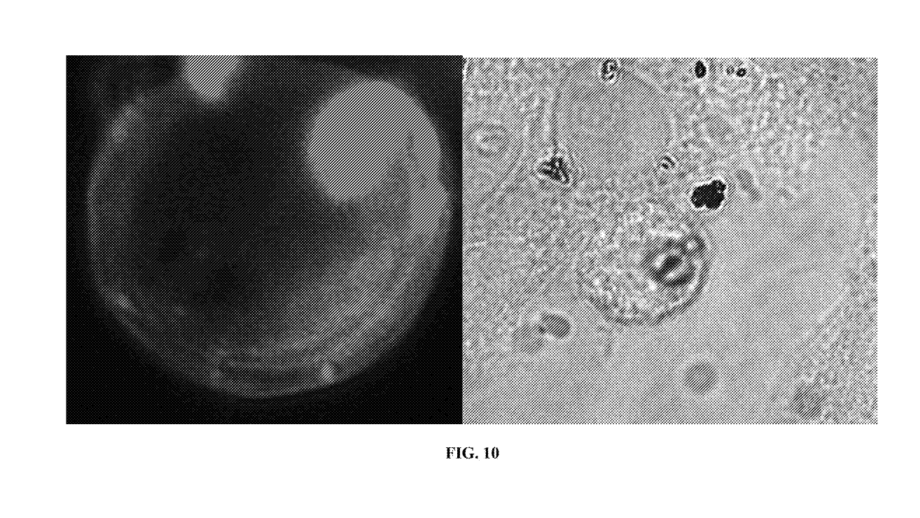

FIGS. 11A-11D—Fluorescent Images of CSMEN penetrated inside HEP2 cells at different focus plane.

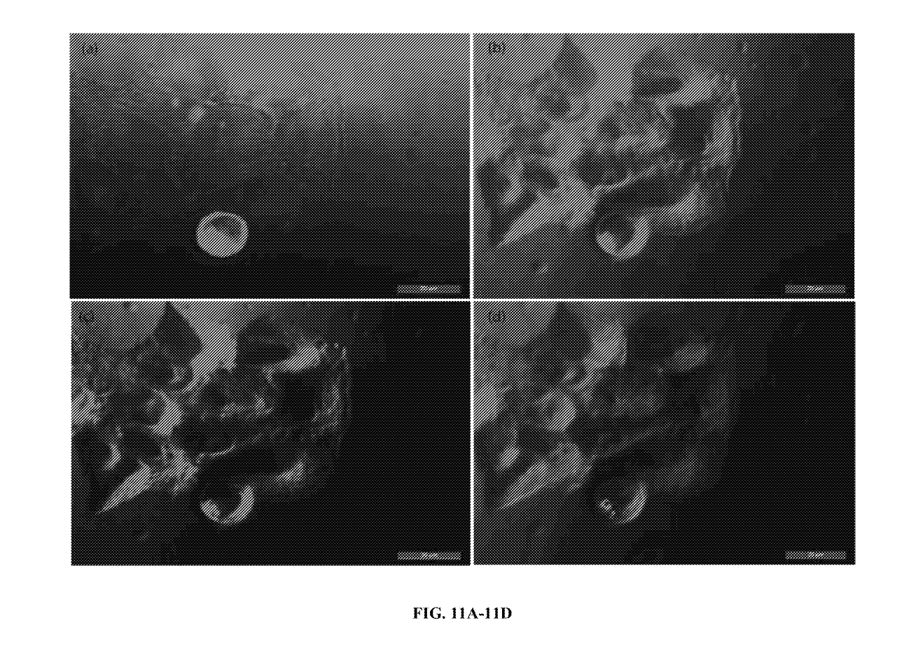

FIG. 12—Real time Images of Magneto-elasto-electroporation (MEEP). Group of CSMENs are travelling from one HEP2 Cell to other.

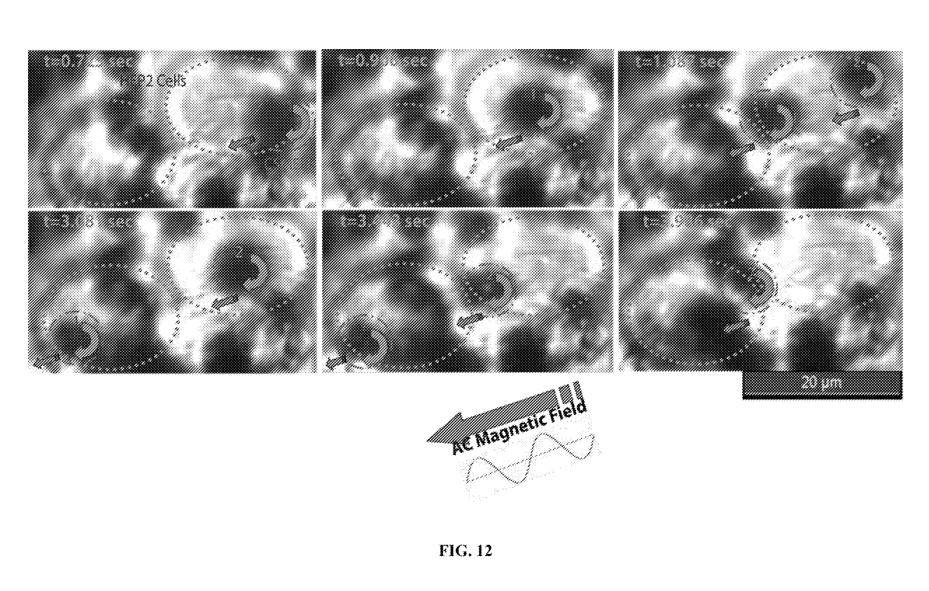

FIGS. 13A-13C—Illustration of certain non-limiting aspects of Magneto-elasto-electroporation (MEEP).

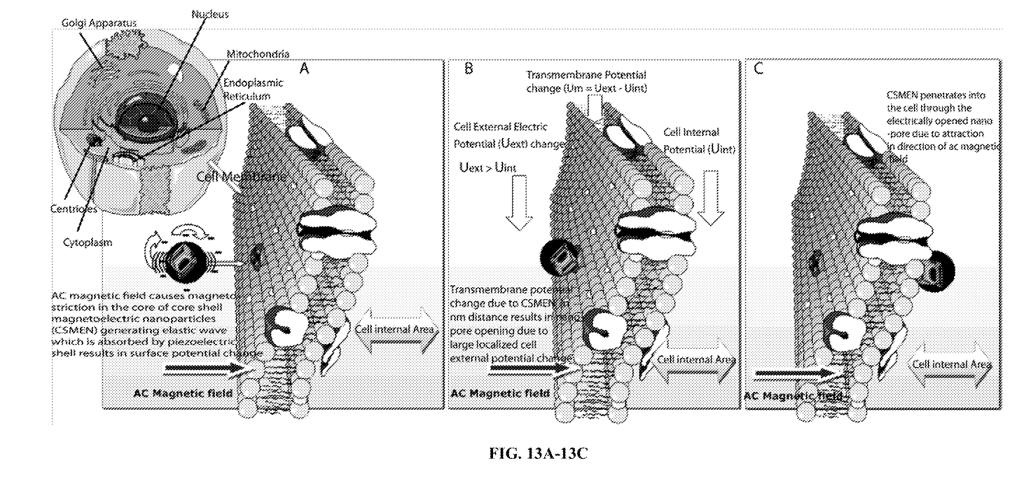

### DESCRIPTION OF ILLUSTRATIVE EMBODIMENTS

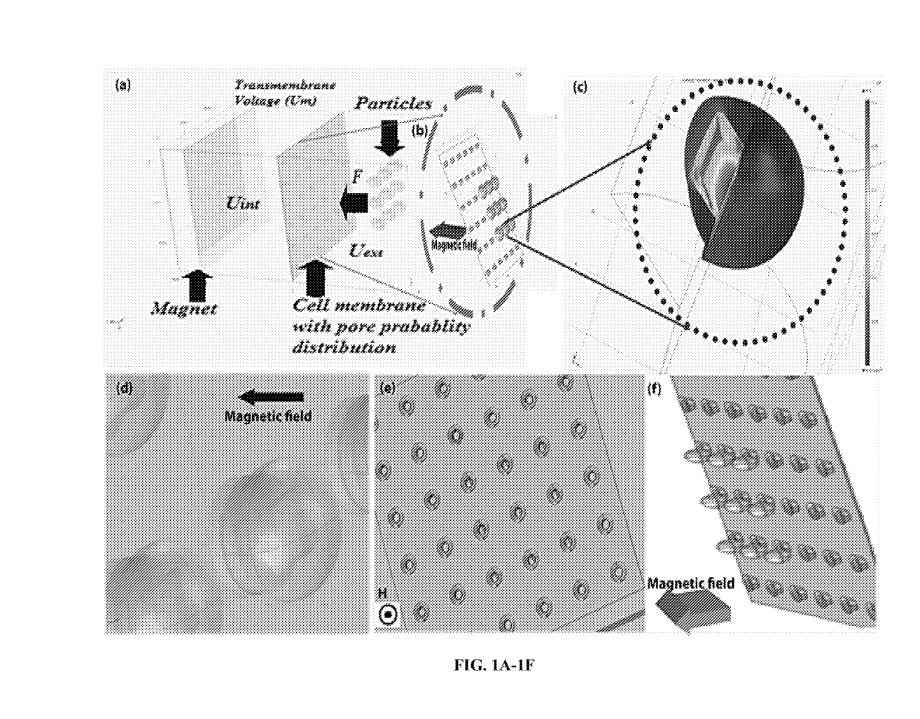

Embodiments of the invention are directed to Magneto-Elasto-Electroporation (MEEP) by electroporation induced by magnetoelectric nanoparticles. To illustrate the MEEP process core (CoFe₂O₄)—shell (BaTiO₃) magnetoelectric nanoparticles (CSMEN) were fabricated, characterized, and used in the studies described herein to provide an example of the compositions and methods for MEEP. Studies were designed and conducted to examine the following issues including: (A) crystallographic phases and multiferroic properties of the core-shell structured magnetoelectric nanoparticles fabricated; (B) influences of DC and AC magnetic field on the CSMEN as function of amplitude and frequency; and (C) cell electroporation phenomenon and its correlation with the magnetic field modulated CSMEN. The detailed experimental analysis demonstrate that the CSMEN retained their physical, electrochemical, magnetic, and piezoelectric properties associated with its respective diffraction patterns, zeta potential, magnetic hysteresis loops, and piezoelectric force microscopic responses. These novel multiferroic properties allow magnetostrictive responses of the cobalt ferrite (CFO, core of the CSMEN) to the externally applied AC magnetic field. Piezoelectric coupling between the core (CoFe₂O₄) and the shell (BaTiO₃) of the CSMEN in turn results in modulation of surface potential of the CSMEN. The combination of Lorentz force and time dependent surface potential, as hypothesized and verified by the inventors, gives rise to the directional movement of the CSMEN and the electroporation of biological cells in the vicinity of CSMENs. An example of the MEEP mechanisms is illustrated using COMSOL Multiphysics software and is presented in FIG. 1.

Core-Shell Magnetoelectric Nanoparticles (CSMEN).

The Core-Shell Magnetoelectric (CSMEN) nanoparticle composites can be synthesized using hydrothermal methods. In certain embodiment the CSMEN are coupled to a cargo moiety that can be transferred across a cell/lipid membrane or similar structure with the aid of the CSMEN. The magnetizable or magnetic core of the nanoparticle can be synthesized or obtained from a commercial source. Shell precursors can be mixed with an appropriate acid in separate containers to obtain citrate solutions of the precursors. These citrates can then be mixed with magnetic core particles in ethylene glycol or similar solvent and heated (e.g., 100° C.) to paralyze the solution. As a result, precursor is efficiently layered on the magnetic core particles. To further stabilize the shell and maintain the integrity, the mixture can be dried and further heated (e.g., 800° C.) in a very low oxygen environment, which prevents oxidation of the magnetic nanoparticles. The dried powder can be repeatedly washed using ethanol and deionized (DI) water and sonicated in ultrasound cleaner to obtain the final crystallized sample of CSMEN nanoparticles.

Core.

A nanoparticle comprising a magnetic material (e.g., a paramagnetic or superparamagnetic material) may include at least one mixed spinel ferrite having the general formula MFe₂O₄, where M is a metal having an oxidation state other than exhibited by the predominant form of iron, which is 3+. Non-limiting examples of M include cobalt, nickel, chromium, gadolinium, zinc, yttrium, molybdenum, bismuth, and vanadium. Metal will be used depending biocompatibility or non-toxicity when administered to a biological sample such as cells or to an organism.

The nanoparticle may be formed by a non-aqueous synthetic route for the formation of monodisperse crystalline nanoparticles, which is described in U.S. Patent Publication No. 2004/00229737 and in U.S. Pat. No. 6,797,380, each of which is incorporated by reference in its entirety. Organometallic precursor materials, such as, but not limited to, transition metal carbonyl compounds, are thermally decomposed in a solvent and in the presence of a surfactant and an oxidant. The organometallic precursors are provided in an appropriate stoichiometric ratio to a nonpolar aprotic solvent containing the surfactant and the oxidant.

A nonpolar aprotic organic solvent can be combined with an oxidant and a first surfactant. The nonpolar aprotic solvent can be thermally stable at the temperatures at which the nanoparticles are formed. In one embodiment, the nonpolar aprotic solvent has a boiling point in the range from about 275° C. to about 340° C. Suitable nonpolar aprotic solvents include, but are not limited to, dioctyl ether, hexadecane, trioctylamine, tetraethylene glycol dimethyl ether (also known as “tetraglyme”), and combinations thereof. The oxidant can comprise at least one of an organo-tertiary amine oxide, a peroxide, an alkylhydroperoxide, a peroxyacid, molecular oxygen, nitrous oxide, and combinations thereof. In one embodiment, the oxidant comprises an organo-tertiary amine oxide having at least one methyl group. One non-limiting example of such an oxidant is trimethyl amine oxide.

The first surfactant optionally can include at least one of a polymerizable functionalized group, an initiating functionalized group, and a cross-linking functionalized group. An amount of the first surfactant is provided to the nonpolar aprotic organic solvent to produce a first concentration of the first surfactant in the nonpolar aprotic solvent. The polymerizable functionalized group may comprise at least one of an alkene, an alkyne, a vinyl (including acrylics and styrenics), an epoxide, an azeridine, a cyclic ether, a cyclic ester, and a cyclic amide. The initiating functionalized group may comprise at least one of a thermal or photoinitiator, such as, but not limited to, an azo compound, a hydroxide, a peroxide, an alkyl halide, an aryl halide, a halo ketone, a halo ester, a halo amide, a nitroxide, a thiocarbonyl, a thiol, an organo-cobalt compound, a ketone, and an amine. The cross-linking functionalized group may be one of a thiol, an aldehyde, a ketone, a hydroxide, an isocyanide, an alkyl halide, a carboxylate, a carboxylic acid, a phenol, an amine, and combinations thereof.

At least one organometallic compound is provided to the combined nonpolar aprotic organic solvent, oxidant, and first surfactant. The at least one organometallic compound comprises at least one metal and at least one ligand. The metal may comprise a transition metal, such as, but not limited to, iron, nickel, copper, titanium, cadmium, cobalt, chromium, manganese, vanadium, yttrium, zinc, and molybdenum, or other metals, such as gadolinium. The at least one ligand may comprise at least one of carbonyl group, a cyclo octadienyl group, an organophosphine group, a nitrosyl group, a cyclo pentadienyl group, a pentamethyl cyclo pentadienyl group, a π-acid ligand, a nitroxy group, and combinations thereof. Non-limiting examples of the at least one organometallic compound include iron carbonyl (Fe(CO)₅), cobalt carbonyl (Co(CO)₈), and manganese carbonyl (Mn₂(CO)₁₀). In one embodiment, an amount of the at least one organometallic compound is provided to the aprotic solvent such that a ratio of the concentration of the at least one organometallic compound to the concentration of the oxidant has a value in a range from about 1 to about 10.

A first organometallic compound can be combined with a nonpolar aprotic organic solvent, oxidant, and first surfactant. The combined first organometallic compound, nonpolar aprotic organic solvent, oxidant, and first surfactant are then preheated under an inert gas atmosphere to a temperature for a time interval. The preheating serves to remove the ligands from the metal cation in the first organometallic compound. The combined first organometallic compound, nonpolar aprotic organic solvent, oxidant, and first surfactant are preheated to a temperature in a range from about 90° C. to about 140° C. for a time interval ranging from about 15 minutes to about 90 minutes.

In another embodiment, the combined nonpolar aprotic solvent, oxidant, first surfactant, and the at least one organometallic compound are heated to under an inert gas atmosphere to a first temperature and maintained at the first temperature for a first time interval. At this point, the at least one organometallic compound reacts with the oxidant in the presence of the first surfactant and the nonpolar aprotic solvent to form a plurality of nanoparticles, wherein each nanoparticle comprises a crystalline inorganic nanoparticle and at least one outer coating comprising the first surfactant, which is disposed on an outer surface of the inorganic nanoparticle and substantially covers and encloses the substantially crystalline inorganic nanoparticle.

The first temperature to which the combined nonpolar aprotic solvent, oxidant, first surfactant, and the at least one organometallic compound are heated is dependent upon the relative thermal stability of the at least one organometallic compound that is provided to the aprotic solvent. The first temperature is in a range from about 30° C. to about 400° C. In one embodiment, the first temperature is in a range from about 275° C. to about 400° C. and, preferably, in a range from about 275° C. to about 310° C. The length of the first time interval may be from about 30 minutes to about 2 hours, depending on the particular organometallic compounds and oxidants that are provided to the aprotic solvent.

In one embodiment, the method may further comprise the step of precipitating the plurality of nanoparticles from the nonpolar aprotic solvent. Precipitation of the plurality of nanoparticles may be accomplished by adding at least one of an alcohol or a ketone to the nonpolar aprotic solvent. Alcohols such as, but not limited to, methanol and ethanol may be used. Alcohols having at least three carbon atoms, such as isopropanol, tend to produce the smallest degree of agglomeration of the plurality of nanoparticles. Ketones such as, but not limited to, acetone may be used in conjunction with—or separate from—an alcohol in the precipitation step.

In another embodiment, the method may also further include a step in which a ligand either partially of completely replaces or is exchanged for the first surfactant in the outer coating. Following the formation of the plurality of nanoparticles, the nanoparticles are precipitated and resuspended in a liquid including a desired ligand (e.g., the neat ligand, or a solution of ligand in a solvent compatible with the existing outer coating). This procedure may be repeated as necessary.

Other methods are described in U.S. Pat. Nos. 6,962,685 and 7,128,891, each of which is incorporated by reference in its entirety, in which nanoparticles are made by treating a mixture of metal salt, alcohol, an acid and amine with ethanol to precipitate magnetic materials.

Shell.

The core can have an overcoating or shell on a surface of the core. The overcoating can be a material having a composition different from the composition of the core. The overcoat of a material on a surface of the nanocrystal can include a substituted, unsubstituted or a mixture of substituted and unsubstituted barium titinate. The shell is basically a ferroelectric material which forms single crystalline coating over the core and can be replaced with any biocompatible ferroelectric material.

Conjugates.

Certain embodiments are directed to nanoparticle compositions and conjugates to facilitate delivery of molecules into a biological system such as cells. The nanoparticles described herein can be directly or indirectly coupled a moiety to be delivered or localized to a cell. The moiety/nanoparticle complex is referred herein as a nanoparticle conjugate or conjugate. The moiety can be permanently coupled to the nanoparticles or reversibly coupled, e.g., the moiety is released from the conjugate at some time after the conjugate is transported across a lipid membrane. The conjugates can impart therapeutic activity by transferring therapeutic compounds across cellular membranes. Certain aspects are directed to nanoparticle agents for the delivery of molecules, including but not limited to small molecules, lipids, nucleosides, nucleotides, nucleic acids, negatively charged polymers and other polymers, for example proteins, peptides, carbohydrates, or polyamines.

In another embodiment, the present invention features methods to modulate gene expression, for example, genes involved in the progression and/or maintenance of cancer or in a viral infection. For example, in one aspect, conjugate can deliver one or more nucleic acid-based molecules to inhibit the expression of the gene(s) encoding proteins associated with pathological conditions or to increase the expression of genes or proteins associated with attenuation of pathological conditions. In certain aspects the pathological condition is, for example, breast cancer, lung cancer, colorectal cancer, brain cancer, esophageal cancer, stomach cancer, bladder cancer, pancreatic cancer, cervical cancer, head and neck cancer, ovarian cancer, melanoma, lymphoma, glioma, or multidrug resistant cancer associated genes.

In a further embodiment, the described molecules can be used in combination with other known treatments to treat conditions or diseases discussed above. For example, the described molecules can be used in combination with one or more known therapeutic agents to treat breast, lung, prostate, colorectal, brain, esophageal, bladder, pancreatic, cervical, head and neck, and ovarian cancer, melanoma, lymphoma, glioma, multidrug resistant cancers, and/or HIV, HBV, HCV, CMV, RSV, HSV, poliovirus, influenza, rhinovirus, west nile virus, Ebola virus, foot and mouth virus, and papilloma virus infection.

Magneto-Elasto-Electroporation (MEEP).

Magneto-Elasto-Electroporation (MEEP) can be expressed and evaluated through following mechanisms:

(i) Magneto-Acoustic Emission—elastic waves generated in acoustic range by core of CSMEN: Exposure to a time-varying magnetic field produces longitudinal lattice vibrations in the core of CSMEN that, in turn, generates elastic waves (Mohaideen and Joy, 2014; Sablik, 2002; du Tremolet de Lacheisserie, 1982). The elastic waves within the magnetostrictive or magnetoelastic material are accompanied by magnetic flux that can be detected remotely. The resonance frequency and amplitude of such vibrations detected depend not only on the nanoparticle materials but also on the surrounding medium that exerts a damping force to the ferromagnetic core oscillations. The fundamental resonant frequency of the cobalt ferrite nanoparticles is described as (Liang and Prorok, 2008)

$${f}_{r} = \frac{1}{2 π d} \sqrt{\frac{H}{ρ ( 1 - σ )}} \qquad Eq . 1$$

Where, H is the amplitude of the applied magnetic field H₀*sin(ωₘt), σ is Poisson's ratio, ρ is the density, and d is the diameter of the CFO particles (considered to be approximately spherical). The applied magnetic field frequency fₐ (fₐ=ωₘ/2π) is in the range of 10-1000 Hz. The initial resonance frequency fₒ of a magnetoelastic particle of mass m₀ demonstrates a decrease (Landeu, 1986) when subjected to a mass loading of Δm due to BaTiO₃ coating:

$$Δ f = {f}_{0} ( \frac{Δ m}{2 {m}_{0}} ) \qquad Eq . 2$$

The shift in resonance frequency Δf is also related to the damping effect of the medium surrounding the nanoparticles of viscosity η and density ρₗ (Stoyanov, 2000):

$$Δ f = \frac{1}{2 ρ d} \sqrt{\frac{η {ρ}_{l} {f}_{0}}{π}} \qquad Eq . 3$$

(ii) Zeta Potential and Magnetoelectric Voltage of the CSMEN—calculation of magnetically controlled surface (zeta) potential change of nanoparticles, due to absorption by the BaTiO₃ shell of acoustic wave created by the core. The electric field generated by each particle on its surface change the transmembrane voltage of the cell which is equal to the difference between external and internal voltage of cell (Uₘ=Uₑₓₜ−Uₙᵢₜ).

(iii) Asymptotic Smoluchowski equations: the asymptotic Smoluchowski equations described in (Krassowska, 2007; Li et al. 2013; Vasilkoski, 2006) for membrane polarization change (that results in opening of nano-pores) can define approximately the radius rⱼ of the electrically opened nano-pores:

$$\begin{matrix} \frac{{dr}_{j}}{dt} = U ( {r}_{j} , {V}_{m} , {σ}_{eff} ) , j = 1 , 2 , … , K , & Eq . 4 \\ U ( {r}_{j} , {V}_{m} , {σ}_{eff} ) = \{ \frac{{V}_{m}^{2}}{1 + {r}_{h} / r + {r}_{t}} + 4 {β ( \frac{{r}_{*}}{r} )}^{4} \frac{1}{r} - 2 π γ - 2 π {σ}_{eff} r \} , \\ in r \geq {r}_{*} & Eq . 5 ( a )\end{matrix}$$

Where, U is the advection velocity. The first term in Equation 5,

$$\frac{{V}_{m}^{2}}{1 + {r}_{h} / r + {r}_{t}} ,$$

accounts for the electric force induced by the local transmembrane potential Vₘ(t, u); the second term

$$4 {β ( \frac{{r}_{*}}{r} )}^{4} \frac{1}{r} ,$$

accounts for the static repulsion of lipid heads; the third term 2πγ accounts for the line tension acting on the pore perimeter; and the fourth term 2πσ_{eff}r accounts for the surface tension of the cell membrane. All parameters of each term are defined in Table 1. The last term contains the effective tension of the membrane σ_{eff}, which is a function of Ap, the combined area of all pores existing on the cell (Neu and Krassowska, 2003),

$${σ}_{eff} ( {A}_{p} ) = 2 {σ}^{′} - \frac{2 {σ}^{′} - {σ}_{0}}{{( 1 - {A}_{p} / A )}^{2}} \qquad Eq . 5 ( b )$$

Where, A_{P}=Σⱼ₌₁^{K}πrⱼ², and A is the surface area of the cell. σ₀ is the tension of the membrane without pores and σ′ is the energy per area of the hydrocarbon-water interface, as defined in Table 1.

TABLE 1
Cell Parameters for Asymptotic Smoluchowski equations-
calculation for pore nucleation characteristics.

| Sign | Value | Definition and References |
|---|---|---|
| A | 15 ∠m | Cell Radius of Human Epithelial Cell |
| Cₘ | 10⁻¹² F m⁻² | Surface capacitance of the membrane |
| | | Hibino et al., 1993) |
| β | 1.4 × 10⁻¹⁹ J | Steric Repulsion Energy (Neu & |
| | | Krassowska, 1999) |
| γ | 1.8 × 10⁻¹¹ | Edge Energy (Glaser, et al., 1988; |
| | Jm⁻¹ | Freeman, et al., 1994) |
| rₕ | 0.97 × 10⁻⁹ m | Constant in Eq. 5 for advection |
| | | velocity (Neu, et al., 2003) |
| rₜ | 0.31 × 10⁻⁹ m | Constant in Eq. 5 for advection |
| | | velocity (Neu, et al., 2003 |
| r* | 0.51 × 10⁻⁹ m | Minimum Radius of Hydrophillic |
| | | pores (Glaser, et al., 1988) |
| H | 5 × 10⁻⁹ m | Membrane thickness (Glaser, et al., |
| | | 1988) |
| σₒ | 1 × 10⁻⁶ J m⁻² | Tension of Bilayer without pores |
| | | He'non, et al., 1999) |
| σ | 2 × 10⁻² J m⁻² | Tension of Hydrocarbon-water |
| | | interface Israelachvili, 1992) |
| Cₘ | 10⁻² F m⁻² | Surface capacitance of the membrane |
| | | (Hibino, et al., 1993) |
| gᵢ | 2 S m⁻² | Surface conductance of the membrane |
| | | (Hibino, et al., 1993) |

(iv) Magnetic moment of nanoparticles: The magnetic moment of a magnet is a quantity that determines the torque it will experience in an external magnetic field which is proportional to the forward movement velocity of particles due to attraction of particles towards magnet in high and low degree of freedom. The kinetics is time and frequency dependent and can define the time of CSMEN penetration into the biological cell.

Forward motion of particles due to attraction force exerted by the magnets can be calculated by magnetization curves. Ferromagnetics such as CoFe₂O₄ nanoparticles or CSMEN multiferroic nanoparticles are complex physical objects since both quantum and classical degrees of freedom have to be taken into account to describe their behaviour in external AC magnetic field. As discussed in (Liubimov, 2014), the particle angular frequency ω and tensor of inertia represent the classical degrees of freedom of a nanoparticle. The tensor of inertia represented by I is considered for a spherical ferromagnetic nanoparticle. The quantum degrees of freedom are described by a macro-spin S. S in the quasi-classical approximation that is defined as the ratio of the particle total magnetic moment to the gyro-magnetic ratio γ, S=−Mₛ V α/γ, where Ms is the saturation magnetization, α is the unit magnetization vector and V is the particle volume. According to the quantum mechanical principle, the total momentum of the particle J is the sum of the mechanical angular momentum, L=Iω, and the total spin momentum S, is conserved for an isolated nanoparticle like the example given in (Liubimov, 2014).

J=L+S=Iω−MₛVα/γ Eq.6

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Crystallographic Phases and Multiferroic Properties of the CSMEN

Synthesis of BaTiO₃ Coated CoFe₂O₄ Nanoparticles:

The Core-Shell Magnetoelectric (CSMEN) nanoparticle composites were synthesized using hydrothermal methods. The CoFe₂O₄ nanoparticles used were obtained from commercial Alfa Aesar Inc. Barium Carbonate (BaCO₃) and Titanium Iso-propoxide (Ti(OCH(CH₃)₂)₄) were mixed with citric acid in separate containers to obtain the Ba and Ti citrate solutions. These citrates were then mixed with CoFe₂O₄ nanoparticles in Ethylene Glycol and heated at 100° C. to paralyze the solution. As a result, barium titanate is efficiently layered on CoFe₂O₄ nanoparticles. To further stabilize the barium titanate shell and maintain the integrity, the mixture is dried and further heated at 800° C. for 8 hour in very low supply of oxygen to prevent oxidation of the ferromagnetic nanoparticles. Finally the dried powder was repeatedly washed using Ethanol and DI water and sonicated in ultrasound cleaner to obtain the final crystallized sample of BaTiO₃ coated CoFe₂O₄ nanoparticles.

EDX and TEM Diffraction Pattern Analysis:

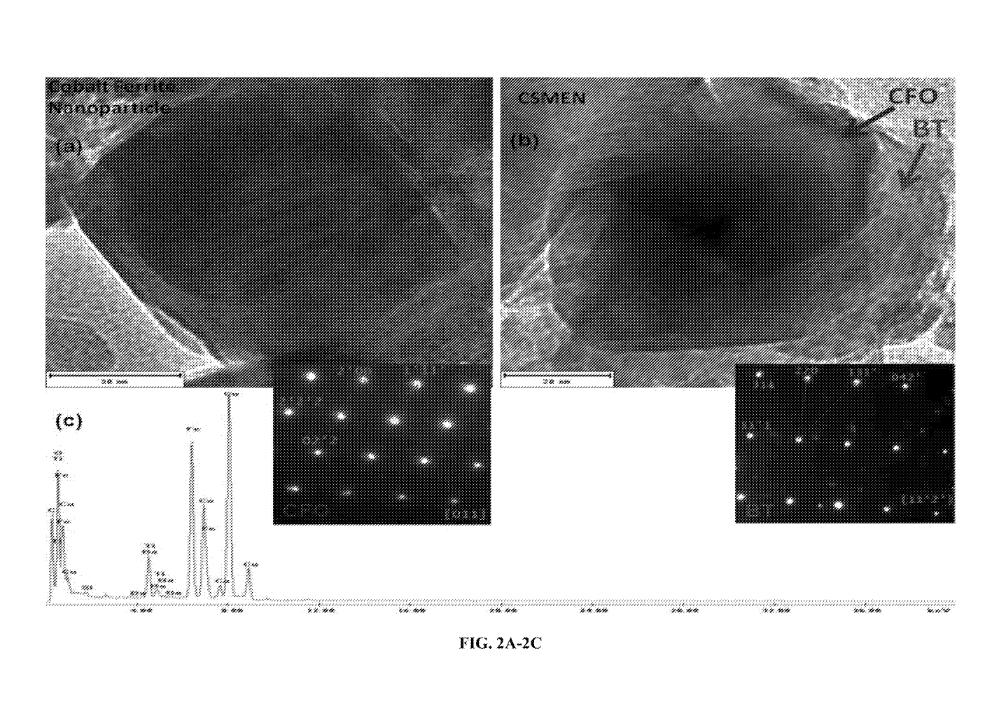

In order to extract further morphological information about the particles, they have been observed under electron microscope. Transmission Electron Microscopy image were taken with a TEM with model no. JEOL2010F and shown in FIG. 2A confirms the core-shell structure; with the core being hexagonal is shape. The size of the nanoparticle as measured from FIG. 2A is approximately ˜80 nm where core is ˜59 nm. To substantiate the single-crystalline nature of the particle, Selected Area Electron Diffraction (SAED) measurements have also been taken. Diffraction patterns shown in FIG. 2A and FIG. 2B corroborates to the single crystalline nature of the shell and core respectively. Gatan Digital Micrograph 1.85 and JEMS have been used to index the diffraction spots and to calculate the zone axis as represented in FIG. 2A and FIG. 2B. Energy Dispersive X-Ray Analysis has also been performed to derive the chemical identity of the particle. As shown in FIG. 2C energy peaks of Barium, Titanium, Cobalt, Iron, and Oxygen peaks are quite prominent. Peaks of Copper and Carbon that are also visible in FIG. 2C are associated with the grid used for the measurement.

Size Analysis:

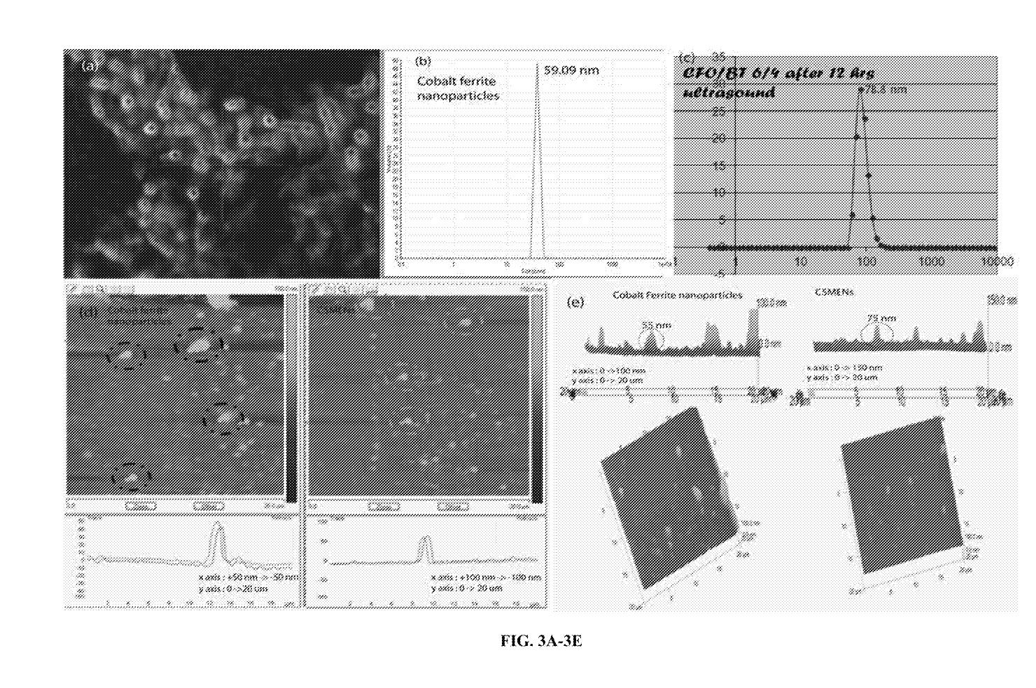

For the size Analysis of CSMEN, measurements were made using dynamic light scattering (DLS) using a Zetasizer Nano ZS (Malvern Instruments, UK). 50 μg/ml concentration of both cobalt ferrite nanoparticle and CSMEN mixed with DI water was sonicated for 12 hours. The solution was analyzed by putting it into the plastic zeta cell. FIG. 3B shows the DLS Size measurement of CFO nanoparticles and FIG. 3C shows the DLS size measurement of BaTiO₃ coated Cobalt Ferrite nanoparticles (CSMEN). The cobalt ferrite nanoparticle's size was measured as 59.09(±05) nm and that of CSMEN size was 78.8(±05) nm. For further confirmation on the size of the nanoparticles and the size of the BaTiO₃ coating, AFM measurements were done. As shown in FIG. 3D, both scanning and 3-D topography image of the CFO nanoparticles and CSMEN on positively charged atomically smooth mica surface shows that CFO nanoparticles are having a size approximately around 50-55 nm. CSMEN size is around 75-80 nm. Microscopy image of AFM in FIG. 3A also shows the coating on cobalt ferrite nanoparticles. Hence the AFM data and the meta zeta sizer data are in good agreement.

Surface/Zeta Potential Measurements:

Zeta Potential measurements were done using Zetasizer Nano ZS using Disposable Capillary Cell (DTS1070). The magnitude of the zeta potential indicates the degree of electrostatic repulsion between adjacent, similarly charged particles in a dispersion (Greenwood, 1999; Hanaor, 2012). Zeta potential measurement illustrates that the surface potential of the nanoparticles changes with the change in content of Barium Titanate coating. Table 2, shows the surface/zeta potential of cobalt ferrite nanoparticles and CSMEN with different weight percentage of CFO and BT as (50-50)%, (60-40)%, and (70-30)% respectively. The Zeta-Potentiometer results show that the CFO nanoparticles possess negative charge on its surface and also after BaTiO₃ coating.

TABLE 2
Zeta Potential/Surface Potential of nanoparticles

| Composition | CFO %100 | CFO-BT % (50/50) | CFO-BT % (60/40) | CFO-BT % (70/30) |
|---|---|---|---|---|
| Zeta Potential | (−)13.46 mV | (−)8.21 mV | (−)24.28 mV | (−)18.904 mV |

PFM Studies—Single Crystalline State of BaTiO₃ Shell:

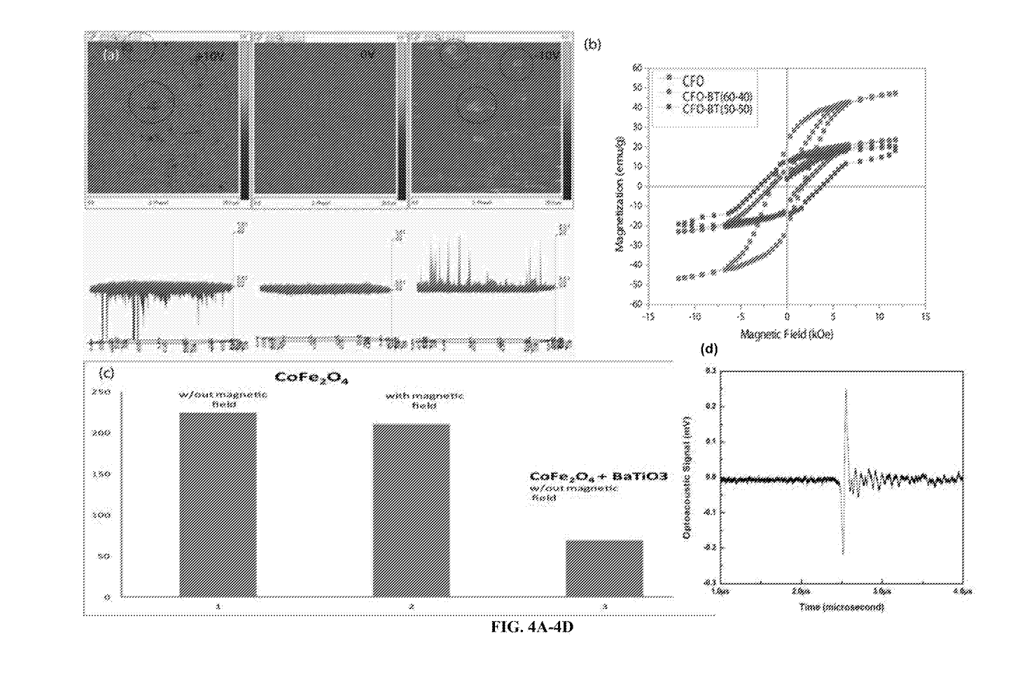

PFM samples were prepared as described in Jaroslaw Grobelny et al. Size Measurement of Nanoparticles Using Atomic Force Microscopy. Materials Science and Engineering Laboratory, National Institute of Standards and Technology, 2009. The Piezo Response Force Microscopy measurements were taken by applying 0 V and biased voltage of +10 V and −10 V. PFM results in FIG. 4A clearly shows the phase transition when biased voltage is applied whereas no phase change can be seen while applying 0 V. This clearly indicates that the barium titanate shell is in single crystalline state and if any pressure is applied from any direction then it will change the surface polarity of the shell of CSMEN. It needs no poling for magnetoelectric voltage output from the CSMEN.

Multiferroic Hysteresis Behavior:

Hysteresis curves were taken to study the change in magnetization of cobalt nanoparticles after coating with barium titanate. The measurements were done by using high sensitivity magnetometer. As shown in FIG. 4B, the magnetometer results shows that cobalt ferrite nanoparticles have 51 emu/g magnetization whereas after coating with different composition of barium titanate the magnetization decreases for example to 22 emu/g with 60% CFO-40% BT and 18.4 emu/g with 50% CFO-50% BT. This decrease in magnetization clearly indicate that Barium titanate layer is coated on the Cobalt ferrite nanoparticles and it is reducing the flux of magnetization in CFO nanoparticles.

### Example 2

### Opto-Acoustic and Magneto-Acoustic Measurements

The influences of DC and AC magnetic fields on the CSMEN as function of amplitude and frequency are further studied by optoacoustic and magetoacoustic measurements.

Using an all optical optoacoustic approach (Yasmin, et al., 2015; Barnes, et al., 2014; Jackson, et al., 1981), pure cobalt ferrite nanoparticles were placed at the bottom of a glass cuvette. The glass cuvette is then filled to the top with liquid (de-ionized water) (˜4 ml). As the cobalt ferrite nanoparticle's density is higher than the water (1 g cm⁻³) cobalt ferrite nanoparticle were then found at the bottom of the cuvette. An optical parametric oscillator (OPO) (EKSPLA model 342NT) laser system pumped by Nd:YAG pulsed laser at 355 nm. A beam at 520 nm was used as an excitation source with a pulse duration of 3.6 ns and a repetition rate of 10 Hz, each pulse having a top hat profile. Energy of the laser was monitored during the duration of the experiment and kept constant at ˜23±1 mJ pulse⁻¹. Upon focusing this pulse energy corresponds to a fluence of ˜2 J cm⁻². On the exposure to the pulsed nanosecond Nd-YAG laser. Upon pulsed excitation, a thermal expansion is produced as a result of light absorption by the nanoparticles which in turns creates a pressure (acoustic) wave capable of travelling through the acoustically coupled medium such as water. To measure this acoustic wave, a 5 mW probe beam from HeNe-laser was passed through the water and just above the nanoparticles. The resulting acoustic wave transiently changes the refractive index of the water which deflects the probe beam from its original optical path. The deflection is measured by a four-quadrant position sensitive detector. On exposure to pulsed laser, cobalt ferrite nanoparticles produce a high Opto-acoustic(OA) wave. However when an AC magnetic field (50 Oe and 60 Hz) was applied, there was an attenuation in Opto-acoustic(OA) emission, which suggest that there is an acoustic emission in the AC magnetic field produces interference with the opto-acoustic emission. Furthermore, when CSMEN were placed in the measurement cuvette with DI water, the OA peaks decreases substantially, which shows that barium titanate shell significantly alters the acoustic wave. Since BT shell is in single crystalline nature, it may affect the potential at surface. FIG. 4C shows the opto-acoustic emission peak intensity of 235.2V/J for cobalt ferrite nanoparticles which decreases to 210V/J when AC magnetic field is applied and further when CSMEN were analysed the PA peak further reduces to 68.76667 V/J. In general, the basic mechanism of magnetoelastic acoustic emission is generation of acoustic emission pulses driven by magnetostriction (Kusanagi et al., 1979; Heaps 1930; Heaps 1941; Grimes 2011) and those originating from local sources of magnetostriction strain due to irreversible displacements of 90° (71° and 109°) domain walls. These pulses carry information about changes in the magneto-elastic state of a ferromagnet. Therefore the magneto-acoustic emission effect is determined by both magnetic and elastic properties of ferromagnet. Magnetoelastic vibrations in a magnetoelastic sensor occur when the applied magnetic field is time varying in nature.

Pulses of acoustic emission generated in the process of cyclic magnetization are measured in most cases using piezoelectric transducers (PZTs).

### Example 3

### Biological Analysis of MEEP Effect

Longitudinal Penetration Analysis:

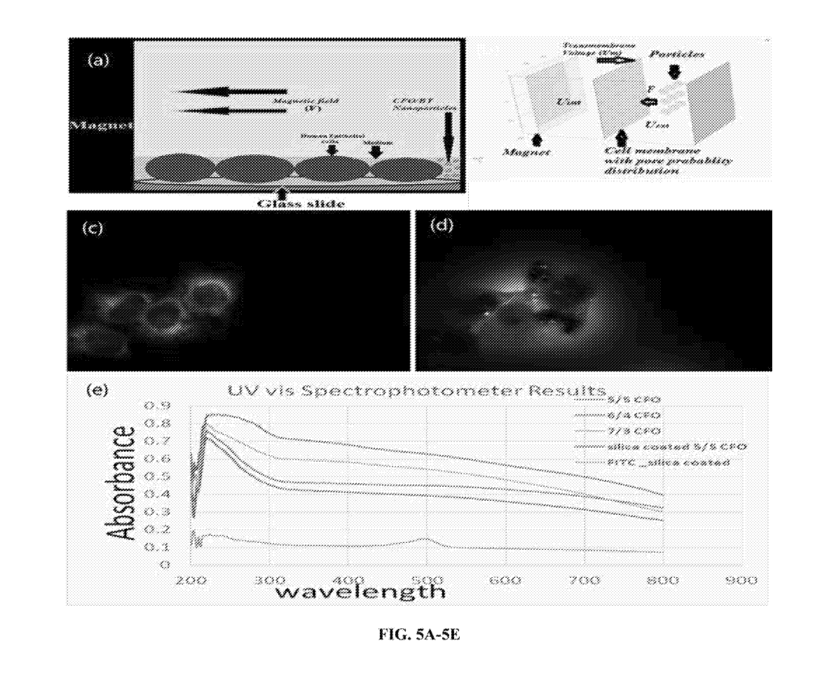

For longitudinal penetration analysis, Human Epithelial Cells HEP2 cells were seeded at the cell density of 1×10⁵ per well in 24 well plate. FITC loaded on silica coated CSMEN (50 μg/ml) was then incubated with the cell and different intensity of AC and DC magnetic field were applied from minutes to an hour. The intensity of DC field varies from 50 Oe-200 Oe and that of AC Magnetic field intensity from 50 to 100 Oe and 60 Hz frequency. FIG. 5A illustrates the experiment setup. CSMEN were added on one end of the well and a magnetic field was applied at the other end. As the HEP2 cells were present in the space between CSMEN and AC magnetic field, the MEEP phenomena happen as the particle in response to applied AC magnetic field creates nanopores in the cell membrane and hence allows the nanoparticle to penetrate through the electrically opened nanopores. FIG. 5B shows the schematics of a study. The cells were then fixed using fixative agent and stained with cell mask for cytoplasm according to the manufacture's protocol. The cells were attached on the glass slide and observed under using fluorescence microscope and confocal microscope. FIG. 5C shows fluorescence microscope image. FIG. 5E shows the UV Vis spectrophotometer result which confirms FITC loading on the silica coated CSMEN.

Fluorescence and Confocal Microscopy:

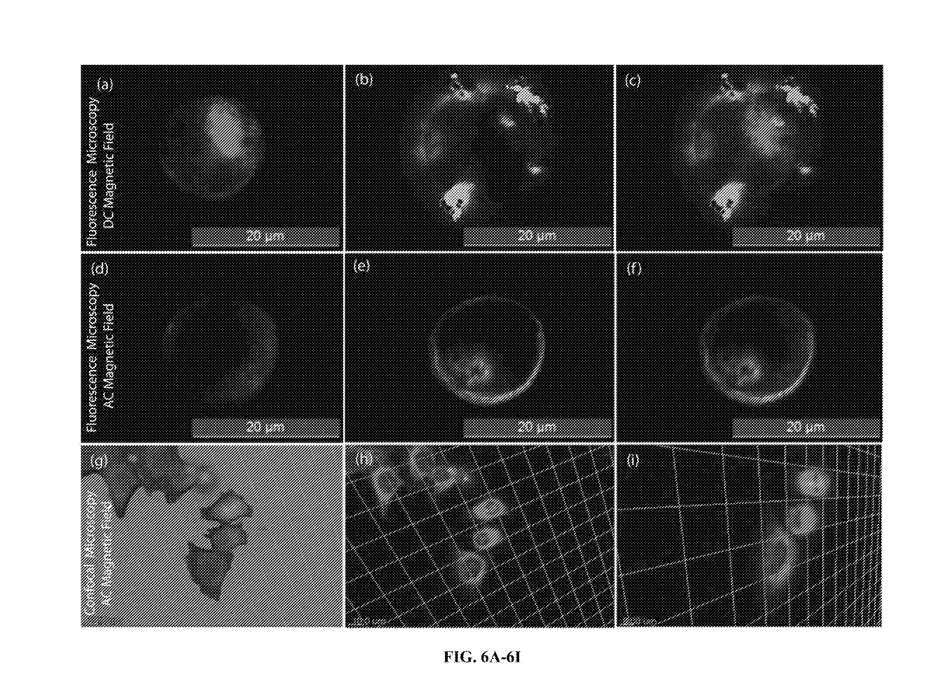

Fluorescence microscopy and confocal images were taken, where all the images were merged using ImageJ software. In presence of DC magnetic field, CSMEN were observed to be outside of HEP2 cell membrane as shown in FIGS. 6A, 6B, and 6C. In the fluorescence microscopy images, the green fluorescence at the cell periphery indicates that the particles did not penetrate the cell membrane in presence of DC magnetic field. Instead they tend to accumulate outside the cell membrane which corroborates the inventors hypothesis. When HEP2 cells with CSMEN were exposed to AC magnetic field for various time periods, green fluorescence was observed within the cell periphery and scatters the green fluorescence with membrane as the scattering boundary. This is shown in FIGS. 6E, 6F and 6G that indicates that the CSMEN penetrates into the HEP2 cells. This penetration of CSMEN into HEP2 cells was further confirmed by confocal microscopy as shown in FIGS. 6G, 6H and 6I.

Transverse Penetration Analysis:

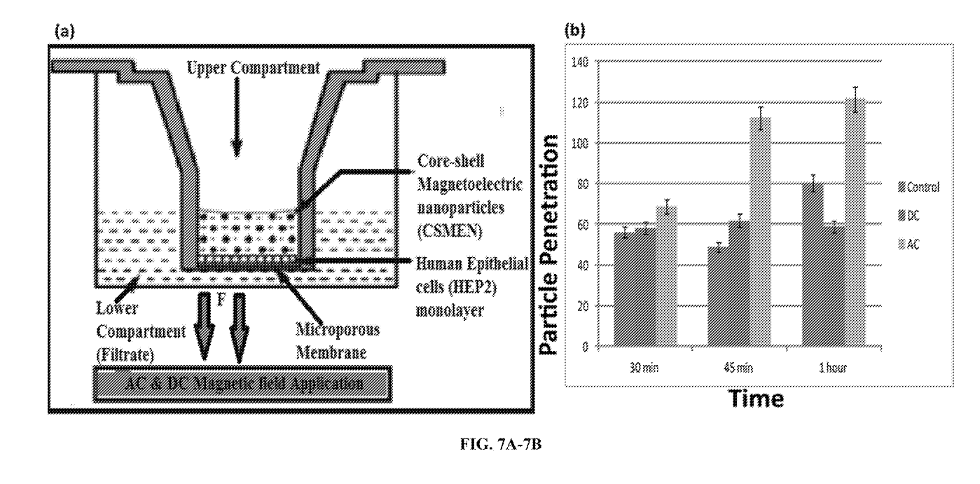

To analyze the penetration of CSMEN into HEP2 cells in presence of AC magnetic field, transwell experiments were performed as discussed in Xue et al, (2013, Int J Biol Sci, 2013. 9(2):174-89). The penetration of nanoparticles into cells was evaluated by using polyethylene teraphthalate (PET) coated control cell culture insert with 1 micron pore diameter. Control inserts in a 24 well plates was first seeded with HEP2 cells at cell density 1×10⁵ per inserts and 500 μl of phosphate buffer solution was added at the bottom of each well with control inserts. After the cells were grown to 100% confluence, the media was replaced with fresh media containing FITC conjugated CSMEN nanoparticles and was incubated at 30, 45, and 60 min. Cells without any CSMEN were used as a negative control and cells with particles but no external magnetic field was used as a control. The supernatant as well as the filtrate were collected and the fluorescence intensity at 490 nm was determined using BioTek micro plate reader. A schematic of the transwell experiment and longitudinal penetration analysis is shown in FIG. 7A. Transwell graph in FIG. 7B shows increased filtrate intensity over time in presence of AC magnetic field whereas the fluorescence intensity of filtrate in case of DC field and control remains minimal. This data suggests that in presence of AC field, more CSMEN enters and passes through the cell due to magneto-elasto-electroporation (MEEP) effect. However, in the case of DC electric field, despite forward movement of the particles due to Lorentz field effect, an absence of MEEP effect prevents the CSMEN to enter inside the cells.

### Example 5

### Methodology

Cyto-Toxicity Test.

MTS assay was performed for cytotoxicity test using epithelial cell line Hep2. Briefly, 10,000 cells were seeded in each well in 96 well plate with 100 μl of culture media. After 24 hour, media was replaced with media containing the samples in different concentration. The concentration used were 2 μg/ml, 10 μg/ml, 20 μg/ml, 50 μg/ml, 100 μg/ml, 200 μg/ml, 500 μg/ml and 1 mg/ml. The cells with samples were incubated for 24 hour. The media is replaced with 100 μl of fresh media and 20 μl of MTS solution was added to each well. After incubating for 4 hour, absorbance at 490 nm was measured using Biotek Plate reader.

The MTS [3-(4,5-dimethylthiazol-2-yl)-5-(3-carboxymethoxyphenyl)-2-(4-sulfophenyl)-2H-tetrazolium] tetrazolium compound is bioreduced by metabolically active cells in to a colored formazan product that is soluble in tissue culture medium. This conversion is accomplished by NADPH or NADH produced by dehydrogenase enzymes in metabolically active cells.

Sample for AFM and PFM Measurements:

PFM measurement on nanoparticles is very complex and needs a substrate with atomically smooth surface (where surface roughness is very low/lower that the particle size-in nanometers). Moreover, particles must stick to the surface of the substrate and should be immobilized so that voltage can be applied using PFM tip and tip can scan the nanoparticles at the same spot as it was on each scan. As shown in Kusanagi (1979, J. Appl. Phys, 50(4):2985-87) to achieve this a Mica substrate was used and cleaved its surface multiple times (4-5 times) by using adhesive tape. With this process an atomically smooth surface was achieved. The cleaved mica substrate was carefully immersed in a mixture of (1:5) Poly-L-Lycin and DI water for 25 mins. This process will make the Mica substrate surface positively charged. Since nanoparticles have negative zeta/surface potential, the nanoparticles stick to the surface of Mica and remain immobilized. Thus both AFM and PFM scanning can be done efficiently.

Silica Coating on CSMEN:

Silica coating on previously synthesized particles was achieved via sol-gel method. As prepared BaTiO₃ coated CFO nanoparticles (10 mg) were suspended in ethanol. The pH of the suspension was adjusted to 10 using 0.1M NaOH to stabilize the particle and to catalyze the sol gel reaction. Under magnetic stirring, 250 μl of Tetraethylorthosilicate (TEOS) was then added to the suspension and allowed to react for 2 hour at 50° C. The hydrolysis and condensation of TEOS forms the silica coating on the surface of the particles. The reaction mixture was then dried overnight to achieve the powder form of the particles.

Cellular Uptake of BaTiO₃ Coated CFO Particles:

The cells were seeded at the density 1×10⁵ per well in 24 well plate. FITC-silica coated CFO particles (50 μg/ml) were then added and various AC and DC field were applied. The cells were then fixed using fixative agent (Poly-L-Lycin) and stained with cell mask for cytoplasm according to the manufacture's protocol. The cells were mounted on the glass slide and examined using florescence microscope and Confocal microscope.

FITC Conjugation on Si Coated CSMEN:

FITC was first conjugated to APTES. Typically, FITC (2 mg) was dissolved in 0.1M APTES in ethanol. The solution was stirred in dark for 24 hour. FITC-APTES (5 ml) solution was then added to silica coated particles (10 mg) and was stirred vigorously for 1 hour. The solution was then incubated for 24 hour at 40° C. The resulting solution was washed repeatedly by ethanol to remove unconjugated FITC.

Manipulation of the biological cell electroporation using core-shell magnetoelectric nanoparticles (CSMEN) in presence of AC magnetic field is described herein. AC magnetic field induced frequency dependent magnetostriction in the core (CoFe₂O₄) of the nanoparticle results in generation of magneto-elastic waves. These elastic waves are coupled as pressure wave by the piezoelectric shell (BaTiO₃) which is in single crystalline state and results in change in surface potential. In nanometer distance from biological cells (Human Epithelial HEP2) this surface potential is very high in mV/nm range. This surface potential change results in external electric field change (Uₑₓₜ) at the outside of the cell membrane, which alters the transmembrane voltage (Uₘ) and affects the cell membrane's nonlinear permeability. The opening of nano-pores in the membrane allows particles of much larger diameters to penetrate through, via an AC driven mechanism that is yet to be fully understood.

The experimental results also indicate that cell membrane's elasticity is influenced by the voltage change at nanometer distance by the particles due to externally applied AC Magnetic field. TEM imaging, DLS measurement and AFM imaging have confirmed the size of CSMEN as ˜78.8 nm with a coating of 19-20 nm of the piezoelectric layer on magnetostricitve cobalt ferrite nanoparticles. PFM measurement has confirmed the single crystalline state of barium titanate shell. Acoustic measurement reveals the opto-acoustic and magneto-acoustic property of cobalt ferrite nanoparticles and absorption of acoustic wave by the BaTiO₃ coating/shell. Fluorescence microscopy, confocal microscopy and transwell experiments recorded the penetration of particle inside the HEP2 when subjected to an external AC magnetic field.

The inventors conclude that CoFe₂O₄—BaTiO₃ CSMEN have the potential to be used as carrier for drug delivery as well as nanoprobe for sensing and electric field application on cells. The DC magnetic field can be used for safe steering of the CSMEN through blood to the infected area and AC magnetic field can be used to trigger MEEP effect. CSMEN loaded with drugs can enter into the infected cell and release payload. Disease treatment as well as sensing can be done simultaneously by exploring the MEEP effect.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Barnes, et al., Probe beam deflection technique as acoustic emission directionality sensor with photoacoustic emission source. Applied optics, 2014. 53(3): p. 511-519.
Chang, D. a. T. S. R., Changes in membrane structure induced by electroporation as revealed by rapid-freezing electron microscopy. Biophys J, 1990. 58(1): p. 1.
Chen, Membrane electroporation theories: a review. Medical and Biological Engineering and Computing, 2006. 44(1-2): p. 5-14.
du Tremolet de Lacheisserie, Magnetoelastic properties of amorphous alloys. J. Magn. Magn. Mater, 1982. 25: p. 251-270.
Freeman, et al., Theory of electroporation of planar bilayer membranes: predictions of the aqueous area, change in capacitance, and pore-pore separation. Biophys J, 1994(67): p. 42-56.
Glaser, et al., Reversible electrical breakdown of lipid bilayers: formation and evolution of pores. Biochim. Biophys. Acta, 1988. 940: p. 275-287.
Greenwood, Electroacoustic studies of moderately concentrated colloidal suspensions. Journal of the European Ceramic Society 1999. 19(4): p. 479-488.
Grimes, et al., Instrumentation and Applications of Magnetoelastic Resonance Sensors: A Review. Sensors, 2011. 11: p. 2809-2844.
Grobelny, et al., Size Measurement of Nanoparticles Using Atomic Force Microscopy. Materials Science and Engineering Laboratory, National Institute of Standards and Technology, 2009.
Hanaor, et al., The effects of carboxylic acids on the aqueous dispersion and electrophoretic deposition of ZrO2. Journal of the European Ceramic Society, 2012. 32(1): p. 235-244.
He'non, et al., A new determination of the shear modulus of the human erythrocyte membrane using optical tweezers. Biophys J, 1999. 76: p. 1145-1151.
Heaps, Discontinuities in Magnetization of Nickel. Phys. Rev, 1941. 59(1): p. 585-587.
Heaps, Discontinuous Change in Length Accompanying the Barkhausen Effect in Nickel. Phys. Rev, 1930. 36(15): p. 326-332.
Hibino, et al., Time courses of cell electroporation as revealed by submicroscopic imaging of transmembrane potential. Biophys J, 1993. 64: p. 1789-1800.
Israelachvili, Intermolecular and Surface Forces. Academic Press, London, U K, 1992. 2nd Ed.
Jackson, et al., Photothermal deflection spectroscopy and detection. Applied optics, 1981. 20(8): p. 1333-1344.
Krassowska & Filev, Modeling electroporation in a single cell. Biophys J, 2007. 92(2): p. 404-17.
Kusanagi, et al., Stress Effect on Magnitude of Acoustic Emission during Magnetization of Ferromagnetic Materials. J. Appl. Phys, 1979. 50, no. 4: p. 2985-2987.
Landau, Theory of Elasticity. Pergamon Press: New York, N.Y., USA, 1986. 3rd ed.
Li, et al., The effect of extracellular conductivity on electroporation-mediated molecular delivery. Biochimica et Biophysica Acta, 2013. 1828: p. 461-470.
Liang & Prorok, Correction for longitudinal mode vibration in thin slender beams. Appl. Phys. Lett, 2007. 90, 221912.
Liubimov, Magnetic nanoparticle traveling in external magnetic field. MISM-2014 Conference, 2014.
Mohaideen and Joy, High magnetostriction parameters for low-temperature sintered cobalt ferrite obtained by two-stage sintering. Journal of Magnetism and Magnetic Materials, 2014. 371: p. 121-129.
Neu & Krassowska, Modeling postshock evolution of large electropores. Phys. Rev. E, 2003. 67: p. 1-12.
Neu & Krassowska, Asymptotic model of electroporation. Phys. Rev. E, 1999. 59: p. 3471-3482.
Neu, et al., Electrical energy required to form large conducting pores. Bioelectrochem. Bioenerg, 2003. 60: p. 107-114.
Sablik, Relationship between magnetostriction and the magnetostrictive coupling coefficient for magnetostrictive generation of elastic waves. 2002. 615: p. 1613-1620.
Stoyanov, A remote query magnetostrictive viscosity sensor. Sens. Actuat. B Chem, 2000. 80: p. 8-14.
Tsong, Electroporation of Cell Membranes. Biophys J, 1991. 60(2): p. 297.
Vasilkoski, Membrane electroporation: The absolute rate equation and nanosecond time scale pore creation. PHYSICAL REVIEW E, 2006. 74, 021904
Xue, et al., A Novel Brain Neurovascular Unit Model with Neurons, Astrocytes and Microvascular Endothelial Cells of Rat. Int J Biol Sci, 2013. 9(2): p. 174-189.
Yasmin, et al., In vitro monitoring of oxidative processes with self-aggregating gold nanoparticles using all-optical photoacoustic spectroscopy. Biosensors and Bioelectronics, 2015. 64: p. 676-682.

What is claimed is:

1. A core-shell magnetoelectric nanoparticle (CSMEN) comprising:
(i) a magnetostricitve core; and
(ii) a ferroelectric shell,
wherein the core is encapsulated by the ferroelectric shell.

2. The nanoparticle of claim 1, further comprising a target moiety.

3. The nanoparticle of claim 1, wherein the core is a CoFe₂O₄ or a substituted CoFe₂O₄ core.

4. The nanoparticle of claim 3, wherein the CoFe₂O₄ or a substituted CoFe₂O₄ core is a single crystalline core.

5. The nanoparticle of claim 1, wherein the shell is a BaTiO₃ shell or a substituted BaTiO₃ shell.

6. The nanoparticle of claim 5, wherein the BaTiO₃ shell or a substituted BaTiO₃ shell is of a single crystalline nature.

7. The nanoparticle of claim 1, wherein the nanoparticle is biocompatible.

8. A method for conducting Magneto-elasto-electroporation (MEEP) comprising:
(i) positioning a nanoparticle of claim 1 within 100 nanometers of a targeted lipid membrane; and
(ii) exposing the target lipid membrane and nanoparticle to an alternating current (AC) magnetic field.

9. The method of claim 8, wherein the AC magnetic field has an intensity between 50 to 100 Oe and a frequency between 20 to 100 Hz.

10. The method of claim 8, wherein the lipid membrane and CSMENs are exposed to the AC magnetic field for 1 to second intervals for between 1 to 60 minutes.

11. The method of claim 8, wherein the lipid membrane is positioned between a magnetic field source and the nanoparticles.

12. The method of claim 8, wherein the nanoparticles are positioned by magnetic steering of the nanoparticles.

13. The method of claim 12, wherein the nanoparticles are administered to a subject.

14. The method of claim 13, wherein the subject is a human.

15. The method of claim 8, further comprising detecting the location of the nanoparticle.
